(19) United States
(12) Patent Application Publication
(10) Pub. No.: US 2017/0112676 A1
(43) Pub. Date: Apr. 27, 2017

(54) EYELID CLOSURE PATCH

(71) Applicant: KMI Surgical, Downingtown, PA (US)

(72) Inventors: Kristen Knepshield Williams, Wayne, PA (US); John A. Price, Palm Coast, FL (US)

(21) Appl. No.: 15/298,281

(22) Filed: Oct. 20, 2016

Related U.S. Application Data
(60) Provisional application No. 62/244,424, filed on Oct. 21, 2015.

(51) Int. Cl. A61F 13/12; A61F 13/02; A61F 13/00

(52) U.S. Cl. CPC A61F 13/124; A61F 13/00059; A61F 13/02; A61F 13/0289

(57) ABSTRACT

An eyelid closure patch including a flexible transparent backing material defining a main body with a width and a length and a tab. An adhesive is applied to one surface of the backing material such that the adhesive substantially covers the main body and the tab. An overlying cover sheet covers the adhesive on the tab to define a non-adhesive tab. Methods of manufacturing an eyelid closure patches are also provided.

This application claims the benefit of U.S. Provisional Patent Application No. 62/244,424, filed Oct. 21, 2015, the contents of which are incorporated herein by reference.

### FIELD OF THE INVENTION

This invention relates to a patch for placing over the eyelids of a patient during surgery. More particularly, there is provided an eye patch which is placed over the patient's eyes during surgery so as to prevent the drying of the eyes by the anesthesia.

### BACKGROUND OF THE INVENTION

General anesthesia reduces the tonic contraction of the orbicularis oculi muscle, causing lagophthalmos i.e. the eyelids do not close fully in 60% of patients.

In addition, general anesthesia reduces tear production and tear-film stability, resulting in corneal epithelial drying and reduced lysosomal protection. The protection afforded by Bell's phenomenon (in which the eyeball turns upwards during sleep, protecting the cornea) is also lost during general anesthesia.

Corneal abrasions are the most common injury; they are caused by direct trauma, exposure keratopathyor chemical injury. An open eye increases the vulnerability of the cornea to direct trauma from objects such as face masks, laryngoscopes, identification badges, stethoscopes, surgical instruments, anesthetic circuits, or drapes. Exposure keratopathy refers to the drying of the cornea with subsequent epithelial breakdown. When the cornea dries out it may stick to the eyelid and cause an abrasion when the eye reopens. Chemical injury can occur if cleaning solutions such as Betadine, chlorhexidine or alcohol are inadvertently spilt into the eye, such as when the face or mouth is being prepped for surgery. Additionally, it is important to protect the eyes during surgery when the patient is placed in the trendelenburg position—tilted so that feet are higher than the head. This position is commonly used for abdominal and gynecological surgery and the eyes must be protected from the patient's own gastric juices flowing into them.

Therefore, the anesthetist must ensure that the eyes are fully closed and remain closed throughout the procedure, in order to avoid exposure keratopathy. Seemingly trivial contact can result in corneal abrasion and the risk of this occurring is markedly increased if exposure keratopathy is already present. Corneal abrasions can be excruciatingly painful in the postoperative period, may hamper postoperative rehabilitation and may require ongoing ophthalmological review and after care. In extreme cases there may be partial or complete visual loss.

Methods to prevent perioperative corneal injuries include simple manual closure of the eyelids, taping the eyelids shut, use of eye ointment, bio-occlusive dressings and suture tarsorrhaphy. However, none of the protective strategies are completely effective; vigilance is always required i.e. the eyes need to be inspected regularly throughout surgery to check they are closed.

For many years, in most western countries, the eyes of patients undergoing general anesthesia have been routinely taped or stuck down with adhesive dressings in an attempt to combat these problems.

Unfortunately, many of the adhesives used on medical products today are temperature and time sensitive i.e. their adhesive strength may increase or decrease when applied to a body temperature and the longer they are applied, the greater the variability in their adhesiveness. What may seem the perfect adhesive strength before application can change as the operation progresses; leading to failure of stick or “over stickiness”. In the former case, the eyelids may move apart and in the latter, may cause bruising, eyelid tears and eyelash removal.

Rolls of tapes are often “laying around” the operating theatre and may not be hygienically clean. Most of these tapes are translucent and so it is not possible to see if the patient's eyes are opened or closed throughout the case. It is not uncommon for the eyelids to move open as the case progresses, even with adhesive tapes stuck onto them.

In a practical sense, these medical tapes/dressings may be difficult to remove from a patient because their ends can become stuck flush with the skin.

While various eyelid closure patches have been developed, the current designs generally provide for a continuous adhesive layer on the eyelid patch or only an adhesive layer about the perimeter of the patch. When the adhesive layer is continuous, it often sticks the wearer's eyelashes and the junction between the upper and lower eyelids, any of which may be tugged when the patch is removed. Conversely, when the adhesive is provided only around the perimeter, the closed eyelid is often not retained and may open, intentionally or inadvertently.

### SUMMARY OF THE INVENTION

In at least one embodiment, the present invention provides an eyelid closure patch including a flexible transparent backing material having a main body with a width and a length and a tab extending from the main body. An adhesive is applied to one surface of the backing material such that the adhesive substantially covers the main body and the tab. An overlying cover sheet covers the adhesive on the tab to define a non-adhesive tab.

In at least one embodiment, the main body has a configuration with a linear edge and an opposed arcuate edge.

In at least one embodiment, an indicia is applied to the tab.

In at least one embodiment, the main body has a length less than 2.5 inches and a width less than 1.25 inches.

In at least one embodiment, the invention provides a method of manufacturing an eye closure patch including positioning an adhesive tape on a cover sheet; cutting through the adhesive tape and the cover sheet to define a perimeter of at least one of the eye closure patches; cutting through the cover sheet to define a first portion of the cover sheet that is a aligned with a main body portion of the eye closure patch and a second portion of the cover sheet that is aligned with a tab portion of the eye closure patch, the second portion of the cover sheet configured to remain adhered to the tab portion to define a non-adhesive tab.

### BRIEF DESCRIPTION OF THE DRAWINGS

The accompanying drawings, which are incorporated herein and constitute part of this specification, illustrate the presently preferred embodiments of the invention, and, together with the general description given above and the detailed description given below, serve to explain the features of the invention. In the drawings:

FIG. 1 is a plan view of exemplary eye closure patches in accordance with an embodiment of the invention.

FIG. 2 is an elevational view along the line 2-2 in FIG. 1.

FIG. 3 is an elevational view along the line 3-3 in FIG. 1.

FIG. 4 is a plan view of an eye closure patch of FIG. 1 removed from the cover sheet.

FIG. 5 is a side elevational view of the eye closure patch of FIG. 4.

FIGS. 6-9 are plan views of exemplary eye closure patches in accordance with alternative embodiments of the invention.

FIGS. 10-12 are top plan views and FIG. 13 is a bottom plan view illustrating steps of an exemplary method of manufacturing eye closure patches in accordance with an embodiment of the invention.

FIG. 14 is a plan view of a strip of exemplary eye closure patches in accordance with an embodiment of the invention.

FIG. 15 is a cross-sectional view along the line 15-15 in FIG. 14.

FIG. 16 is a view similar to FIG. 15 illustrating folding away of the cover sheet.

### DETAILED DESCRIPTION OF THE INVENTION

In the drawings, like numerals indicate like elements throughout. Certain terminology is used herein for convenience only and is not to be taken as a limitation on the present invention. The following describes preferred embodiments of the present invention. However, it should be understood, based on this disclosure, that the invention is not limited by the preferred embodiments described herein.

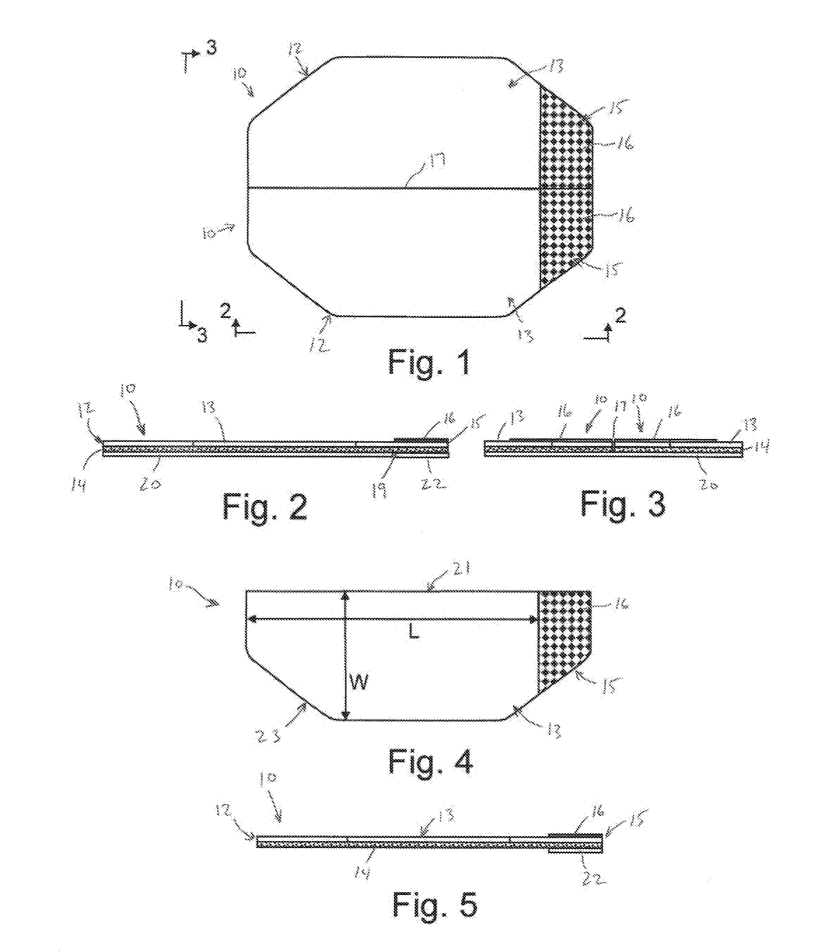

Referring to FIGS. 1-5, an exemplary eye closure patch 10 in accordance with an embodiment of the invention will be described. The eye closure patch 10 generally comprises a flexible transparent backing material 12 which defines a main body 13 and a tab 15 extending from the main body 13. One surface of the backing material 12 is coated with an adhesive 14. In the present embodiment, the adhesive 14 extends along the entire surface of the backing material 12, with the main body 13 including a removable cover sheet 20 such that the adhesive 14 may be exposed as illustrated in FIG. 5 by removing the cover sheet 20. The portion of the backing material 12 defining the tab 15 has an overlying cover sheet 22 applied to the adhesive 14. The overlying cover sheet 22 remains overlying the adhesive 14 such that the tab 15 defines an adhesive free portion to assist in removal of the patch 10. In the illustrated embodiment, the overlying cover sheet 22 is defined by a cut 19 through the cover sheet 20 (see FIG. 2).

In the illustrated embodiment, indicia 16 is provided on the opposite surface of the backing material 12 in the area of the tab 15 to identify the location of the tab 15 to a user. The indicia 16 may be, for example, a printed pattern, word or the like or a coloring of the backing material 12. In the illustrated embodiment, the patches 10 are manufactured such that a pair of patches 10 share a common cover sheet 20, with a cut 17 between the patches 10 (see FIG. 3).

The backing material 12 can comprise any clear medical grade backing material such as paper, woven cloth or non-woven polyesters, foams and polymeric films, such as polyurethane, a polyester, a polyolefin (polyethylene, polypropylene), polyvinyl alcohol, polyvinyl acetate, and the like. The cover sheets 20, 22 or films can be prepared from silicone or polymeric films such as polyolefin polymers, vinylidene copolymers, fluorocarbon films, polyethylene terephthalate, acrylic polymers or the like. The films or other substrates which may be employed in the invention may have a thickness of from 0.0005 to 0.05 inch.

The adhesive 14 is supplied to the backing material 12 in sufficient amount to cause adherence. The adhesive 14 is a transparent pressure sensitive water soluble adhesive which can be easily removed and can be used with comfort by the patient. A natural or synthetic hydrocolloid provides sufficient stickiness to cause the patch to adhere sufficiently during the operation. Suitable synthetic and natural water soluble hydrocolloids and gelatins include karaya gum, guara gum, collagen, polysaccharide gum, locust bean gum, powdered pectin, gelatin, carboxymethyl cellulose and the like. As further examples, the adhesive may be a synthetic rubber, acrylate, silicone or soft silicone gel adhesive or any other suitable medical grade adhesive.

If desired, the pressure sensitive adhesive can include 0 to 10% by weight of the usual modifiers, fillers, extenders, antioxidants, stabilizers and other such ingredients known in the art for inclusion in such compositions. Thus, for example, plasticizers or solvents such as mineral oil or petrolatum may be added to improve adhesive characteristics.

The extenders can include finely divided clays, bentonites, starches or other inert ingredients normally used in adhesive compositions. Antioxidants and stabilizers can be used at levels up to about 3% by weight of the total composition. Suitable oxidants and stabilizers include bitylzimate, 2,6-ditert-butyl-4 methyl phenol sold under the trademark IONOL by Shell Chemical Company, alkylated diphenyl phenols, non-allergenic substances such as zinc oxide can also be used as a stabilizer. The adhesive preferably has a Williams plasticity number of about 1 to 4 mm according to the procedure of ASTM 0926-67 (1978).

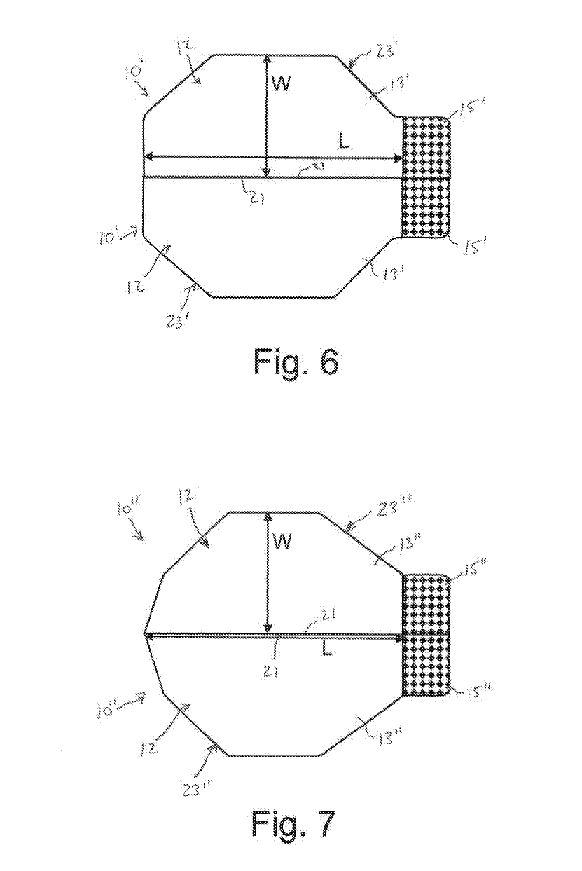

Referring to FIGS. 4 and 5, the main body 13 of each eye closure patch 10 has a partial octagon configuration with a linear edge 21 and an arcuate edge 23 opposite from the linear edge. Arcuate is not limited to a smooth arc, but instead is used to mean an edge which is bowed away from the linear edge. The arcuate edge 23 may comprise a combination of linear and arc sections. FIGS. 6-9 illustrated eye closure patches 10′, 10″, 10′″, 10^{iv} having alternative main body 13′, 13″, 13′″, 13^{iv} and tab 15′, 15″, 15′″, 15^{iv} configurations, but in each embodiment having a linear edge 21 and an arcuate edge 23′, 23″, 23′″, 23^{iv}. In other regards, the eye closure patches 10′, 10″, 10′″, 10^{iv} are generally the same as described in the previous embodiment.

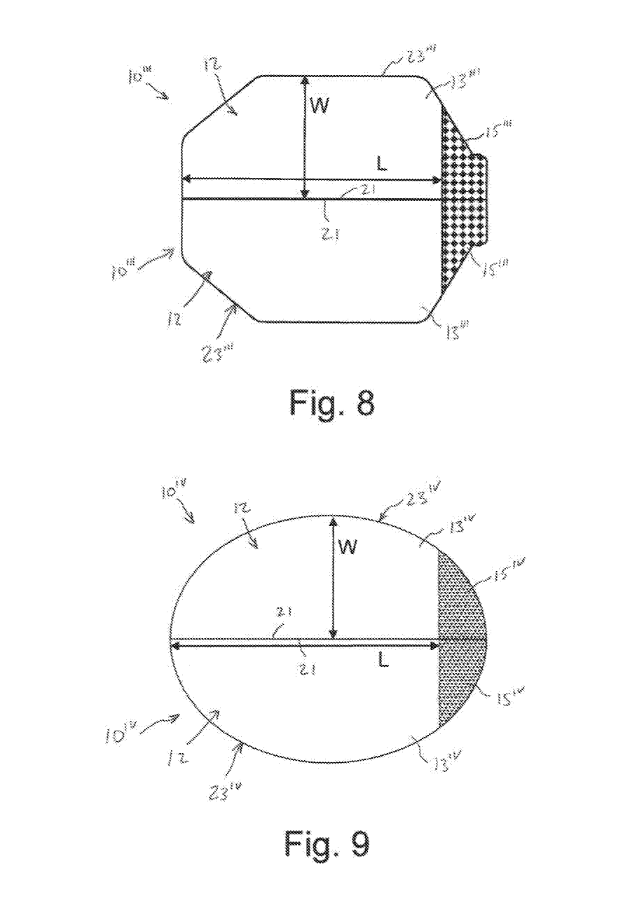

In the embodiment illustrated in FIG. 6, the main body 13′ has a generally octagonal configuration, however, with the central portion thereof being more narrow than the previous embodiment and the tab 15′ extending from the end octagonal wall instead of being formed along the wall as in the previous embodiment. The eye closure patch 10″ illustrated in FIG. 7 is similar to the previous embodiment except that the main body 13″ has an asymmetrical configuration with the portion of the arcuate edge 23″ opposite the tab 15″ extending from the linear edge 21 at an acute angle. The eye closure patch 10′″ illustrated in FIG. 8 also includes a main body 13″ having an asymmetrical configuration with the central portion of the arcuate edge 23′″ extending longer and the portion adjacent the tab 15′″ extending a steeper angle. In the embodiment of the eye closure patch 10^{iv} illustrated in FIG. 9, the main body 13^{iv} and the tab 15^{iv} have a generally hemi-elliptical configuration, with the arcuate edge 23^{iv} having a substantially smooth elliptical arc. It is contemplated that the main body and tab may have a rectangular configuration with the arcuate edge defined by side edges and an opposed edge substantially parallel to the linear edge. The exemplary embodiments are for illustrative purposes and the configuration of the main bodies and tabs are not limited to the illustrated configurations. The specific configuration may be selected such that the eye closure patch 10 provides a desired coverage of the wearer's eye while minimizing potential discomfort to the wearer.

In each of the illustrate embodiments, the main body 13, 13′, 13″, 13′″, 13^{iv} has a length L parallel to the linear edge 21 and a maximum width W perpendicular to the linear edge 21. Preferably, the length L is less than 2.5 inches and the width W is less than 1.25 inches, however, the width W and/or length L could be larger. In one exemplary embodiment, the main body 13, 13′, 13″, 13′″, 13^{iv} has a length L of about 2.125 inches and a width W of about 1.0 inches. With this configuration, when the patch 10 is positioned over the wearer's eye, the adhesive 14 will contact along the wearer's closed eyelid and maintain the eyelid in the closed position.

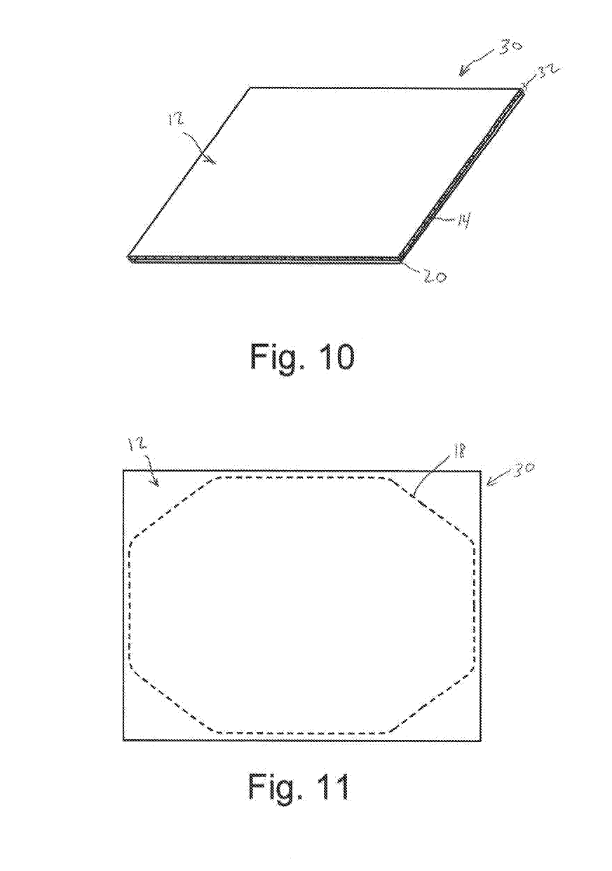

Referring to FIGS. 10-13 an exemplary method of forming the eye closure patches 10-10^{iv} will be described. While the method is illustrated and described with respect to the patch 10, it is applicable to each of the patches 10-10^{iv}. Referring to FIG. 10, a blank 30 for forming a pair of patches 10 includes a piece of tape 32, which defines the transparent backing material 12 and the adhesive 14, applied to a non-adhesive cover sheet 20. The tape 32 may be, for example, 3M™ Blenderm™ Surgical Tape. Referring to FIG. 11, a cut 18 is made through the blank 30 which defines the perimeter of the patch(es) 10. In the illustrated embodiment, the perimeter is for a pair of patches 10 (similar to that shown in FIG. 1), however, it is contemplated that the perimeter may be of a single patch or more than two patches.

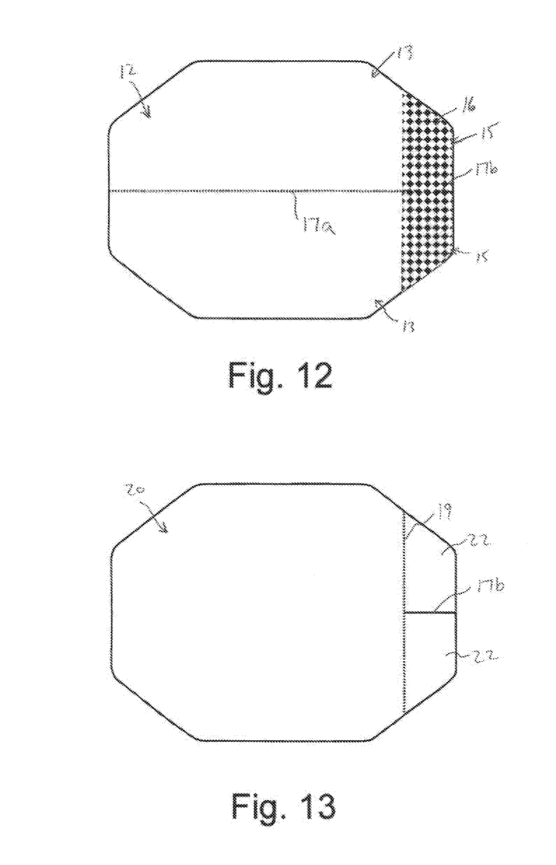

Referring to FIG. 12, the indicia 16 is applied, for example, printed, to the top surface of the backing material 12 to define the tab 15 areas. Also, in the area of the main bodies 13, a partial cut 17a is made through the backing material 12 and adhesive 14, but not the non-adhesive cover sheet 20, to define the two main bodies 13 while allowing both main bodies 13 to remain positioned on the single cover sheet 20. Additionally, in the area of the tabs 15, a full cut 17b extends through the backing material 12, the adhesive 14 and the cover sheet 20 to define two separate tabs 15, each of which will maintain a separate portion of the cover sheet 20 as the respective overlying cover sheet 22. In the event that the perimeter cut defined a single patch, the cuts 17a, 17b would not be necessary.

Referring to FIG. 13, a partial cut 19 is made through the cover sheet 20 but not the adhesive 14 and the backing material 12 to define the overlying cover sheets 22 distinct from the cover sheet 20. As explained above, the overlying cover sheets 22 remain in place, covering the adhesive 14 to define the respective non-adhesive tabs 15.

The process described allows two patches to be manufactured in an efficient, economical manner. While the various cuts 17a, 17b, 18 and 19 and the application of indicia 16 are illustrated in a particular sequence, it is understood that the invention is not limited to such. The cuts and application of indicia may be performed in different sequences and/or steps may be performed simultaneously.

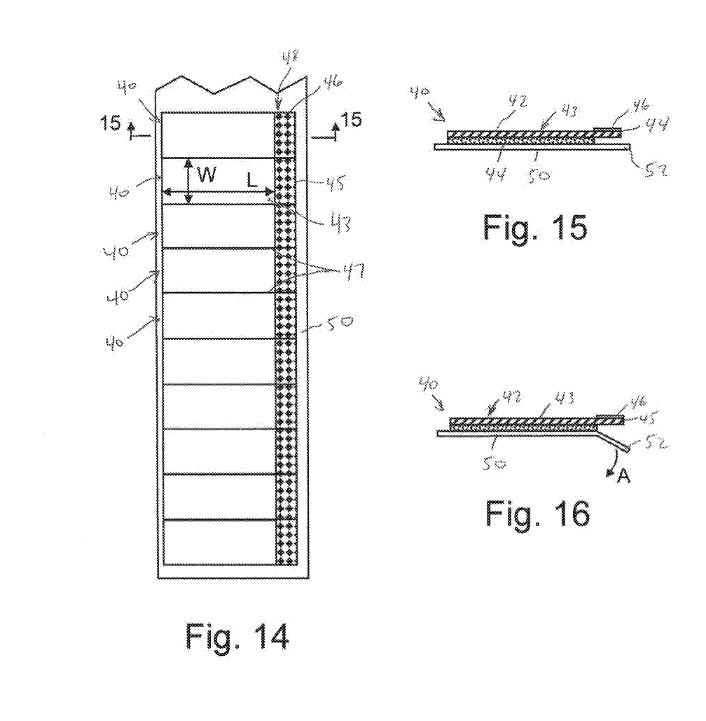

Referring to FIGS. 14-16, a patch 40 in accordance with another embodiment of the invention will be described. The patch 40 generally comprises a flexible transparent backing material 42 which defines a main body 43 and a tab 45 extending from the main body 43. One surface of the backing material 42 is coated with an adhesive 44 along the main body 43 while the tab 45 is free of adhesive to assist in removal of the patch 40. An indicia 46 may be applied to the tabs 45 such that the tabs 45 are identifiable by a user.

The main body 43 has a rectangular configuration with a length L and width W. Preferably, the length L is less than 2.0 inches and the width W is less than 1.25 inches. In one exemplary embodiment, the main body 43 has a length L of about 1.75 inches and a width W of about 1.0 inches. With this configuration, when the patch 40 is positioned over the wearer's eye, the adhesive 44 will contact along the wearer's closed eyelid and maintain the eyelid in the closed position.

The patches 40 are preferably supplied in the form of a strip 48 with each patch 40 being separated at an area with perforations 47 to form a tear strip. The strip 48 is preferably removably positioned on a cover sheet 50 to protect the adhesive 44. The cover sheet 50 preferably has a width greater than that of the strip 48 such that at least one end 52 extends below and beyond the tabs 45. With such a configuration, the end 52 can be easily folded away from the tab 45, as indicated by arrow A in FIG. 16 to allow the tab 45 to be grasped and the patch 40 removed from the sheet 50. For convenience in an operating room, the sheet 50 can be supplied in one continuous roll. Alternatively, each patch 40 may be wrapped separately between opposed protective sheets.

These and other advantages of the present invention will be apparent to those skilled in the art from the foregoing specification. Accordingly, it will be recognized by those skilled in the art that changes or modifications may be made to the above-described embodiments without departing from the broad inventive concepts of the invention. It should therefore be understood that this invention is not limited to the particular embodiments described herein, but is intended to include all changes and modifications that are within the scope and spirit of the invention as defined in the claims.

What is claimed is:

1. An eyelid closure patch comprising:
a flexible transparent backing material defining a main body with a width and a length and a tab;
an adhesive applied to one surface of the backing material such that the adhesive substantially covers the main body and the tab; and
an overlying cover sheet covering the adhesive on the tab to define a non-adhesive tab.

2. The eyelid closure patch according to claim 1 wherein the main body has a configuration with a linear edge and an opposed arcuate edge.

3. The eyelid closure patch according to claim 3 wherein the arcuate edge includes at least one linear section.

4. The eyelid closure patch according to claim 3 wherein the arcuate edge has a combination of linear and arc sections.

5. The eyelid closure patch according to claim 3 wherein the arcuate edge has a continuous arc.

6. The eyelid closure patch according to claim 5 wherein the continuous arc has an elliptical configuration.

7. The eyelid closure patch according to claim 1 wherein the main body has a rectangular configuration.

8. The eyelid closure patch according to claim 1 wherein the main body has a length less than 2.5 inches and a width less than 1.25 inches.

9. The eyelid closure patch according to claim 1 wherein an indicia is applied to the tab.

10. The eyelid closure patch according to claim 9 wherein the indicia is a printed material on the tab.

11. A method of forming an eye closure patch comprising the steps of:
positioning an adhesive tape on a cover sheet;
cutting through the adhesive tape and the cover sheet to define a perimeter of at least one of the eye closure patches;
cutting through the cover sheet to define a first portion of the cover sheet that is aligned with a main body portion of the eye closure patch and a second portion of the cover sheet that is aligned with a tab portion of the eye closure patch, the second portion of the cover sheet configured to remain adhered to the tab portion to define a non-adhesive tab.

12. The method according to claim 11 wherein the perimeter includes two eye closure patches and the method further comprises cutting through the adhesive tape, but not the cover sheet, between the main body portions of two eye closure patches.

13. The method according to claim 12 further comprising the step of cutting through the adhesive tape and the cover sheet between the tab portions of the two eye closure patches.

14. The method according to claim 11 further comprising the step of applying an indicia to the tab portion.

15. The method according to claim 14 wherein the step of applying an includes printing a pattern or word on a non-adhesive side of the adhesive tape.
